(19) United States
(12) Patent Application Publication
(10) Pub. No.: US 2008/0306331 A1
(43) Pub. Date: Dec. 11, 2008

(54) STIMULATION ENHANCEMENT DEVICE

(76) Inventors: Steven C. Morreale, Carmel, NY (US); Una Morreale, Carmel, NY (US)

Correspondence Address: LACKENBACH SIEGEL, LLP, LACKENBACH SIEGEL BUILDING, 1 CHASE ROAD, SCARSDALE, NY, 10583, US

(21) Appl. No.: 11/758,924

(22) Filed: Jun. 6, 2007

(51) Int. Cl. A61F 5/00

(52) U.S. Cl. 600/38

(57) ABSTRACT

A stimulation enhancement device includes a generally elongate main body portion having predetermined length, width and thickness, and defines an upwardly-facing surface and an opposing downwardly-facing surface. The downwardly facing surface is generally flat and said upwardly-facing surface is provided with a plurality of generally transverse protuberances spaced from each other along the length direction of the main body portion and that rise above the upwardly-facing surface. The downwardly-facing surface of the device may be positioned on the upper surface of an erect penis and, when maintained thereon, effectively increases the girth of the penis while the protuberances are effective to provide enhanced vaginal and clitoral stimulation.

### BACKGROUND OF THE INVENTION

1. Field of the Invention

The invention generally relates to stimulation enhancing devices and, more specifically, to a device for enhancing stimulation in a woman during vaginal intercourse without diminishing penile sensitivity in the man.

2. Description of the Prior Art

Numerous sexual aids have been devised. Many of these are applied to or supported on the penis of the male partner during vaginal intercourse. However, most of these are intended to be used as a prosthesis to support the male organ of men that have penile dysfunctions and have difficulty in having an erection or maintaining the erect condition of the penis to enable or promote effective vaginal intercourse. Examples of such penile support devices are disclosed, for example, in U.S. Pat. Nos. 2,018,328; 2,868,192; 2,899,957; 3,131,691; 4,615,337; 4,672,954; 4,893,616; 5,360,390; 5,522,787 and 5,823,939. However, for the most part these may facilitate vaginal intercourse but do not enhance the stimulation of the female during such intercourse.

A distinction is sometimes made between clitoral and vaginal orgasms in women. An orgasm that results from combined clitoral and vaginal stimulation is called a blended orgasm. Many doctors have claimed that vaginal orgasms do not exist and that female orgasms are obtained only from clitoral arousal. Recent discoveries about the size of the clitoris—it extends inside the body, around the vagina- complicate or may invalidate attempts to distinguish clitoral vs. vaginal orgasms. Some women feel inadequate when they cannot achieve orgasm via vaginal intercourse that involved little or no clitoral stimulation. In 1966, Masters and Johnson published pivotal research into the phases of sexual stimulation. One of the results was the promotion of the idea that vaginal and clitoral orgasms follow the same stages of physical response. Additionally, Masters and Johnson argued that clitoral stimulation is the primary source of orgasms.

A new understanding of the “vaginal” orgasm has been emerging since the 1980s. Many women report that some form of vaginal stimulation in concert with clitoral stimulation is essential to experiencing a fully satisfactory orgasm. Recent anatomical research has pointed towards a connection between intra-vaginal tissues and the clitoris—it has been shown that these tissues have connecting nerves. This information, combined with the anatomical evidence that the internal part of the clitoris is a much larger organ than previously thought, could explain credible reports of orgasms in women who have undergone clitorectomy as part of so-called female circumcision (also called female genital mutilation). The discovery of the biological link between the clitoris and the vagina lends further credence to the belief that the clitoris is the ‘seat’ of the female orgasm and indicates that the clitoris is simply far wider-spread than the visible part most people commonly associate with it. It is possible, however, that some women have more extensive clitoral tissues and nerves than others, meaning that some women can achieve orgasm solely via direct stimulation of the external portions of the clitoris.

In view of the foregoing it is evident that for more effective stimulation a woman it may be desirable or indeed preferable to enhance both vaginal stimulation and/or clitoral stimulation-the extent of each perhaps varying from one female to another. A number of devices have been proposed to enhance the stimulation of the clitoris. Typically, whether condoms or separate “enhancement” devices attached to the male member, these normally include some form of protuberance(s) that project upwardly from the upper surface of the penis and positioned to engage the clitoris which is the visible knob-like portion located near the anterior junction of the labia minora, and spaced above the opening of the urethra and vagina. The projections or protuberances are designed to span that spacing to physically engage the clitoris during vaginal intercourse when the penile member is disposed within the vagina. One such device is disclosed in U.S. Pat. No. 4,224,933 which disclosed a sexual aid that stimulates both persons. The device includes a tubular sleeve of thin latex, constrictions at opposite end portions thereof, several stiffening stays imbedded in the sleeve extending longitudinally, a zig-zag row of balls tethered on the outer side of the sleeve, and a band adjustably attached to straps near a base end of the sleeve. The tubular body is made of stretchable, surgical latex or other soft, equivalent material, which is approximately one-sixteenth inch. The body is about three and one-half inches long, and approximately one and three-fourths inches in diameter. A forward end portion of the body is one-sixteenth inch less in diameter than the body long, main portion. The end portion keeps the same snugly on the penis. A diametrically restricted portion, at the other end of the main portion, is of thicker material, and is one-fourth inch wide, while being three-sixteenths inch less in diameter than the main portion, so as to grasp firmly around the base end of the penis. A terminal end portion, adjacent the restricted portion flared outwardly, and has a rolled over edge. While specific dimensions have been indicated for the body, the patentee contemplates that is may be manufactured in other sizes, so as to accommodate all wearers. From five to seven stays, depending on stimulator diameter size, are imbedded within the main portion of the body. The stays, made of semi-rigid plastic, are flat, with all corners rounded, so as not to pierce the body. A row of one-half inch diameter, smooth, soft balls, located outside of the body, are attached to one of the stays by a stem, that is only one-thirty second inch long, in order to allow the balls a limited sway in any direction. Each alternate ball is attached to opposite side edges of the stay, as shown, and may be either of plastic or rubber. Adjustable strap members may be incorporated into the body construction, by affixing each, at its one end, to diametrically opposite sides of the terminal end portion and the opposite ends of the straps adjustably attachable to opposite ends of a rubber, thread-like band, positionable behind the testicles. In operative use, the restricted area of the device gently limits the flow of blood from the penis, due to a slight pressure on this organ at the organ base. The blood, therefore, stays in the penis, creating and helping to maintain a normal, natural erection. The extra blood supply also causes the nerve endings in the penis to be super-sensitive, thus producing unbelievable sensations during the sex act. The rolled over edge is used to enable the user to pull the device into position on the penis, which should be back against the body. When in place, the two rows of balls will center on top of the penis. The light-weight stays keep the device from rolling up the penis, when in use. The restricted area also functions to keep the device from sliding off the penis, while in use. To further insure the device's staying in place, the rubber, thread- like band can be made a part of the unit, and it then can be placed behind the testicles, but as a rule, this is not needed. During the act, the balls massage the clitoris of the female, thus creating pleasant sensations which the male organ is incapable of producing by itself, and thus satisfying the female. When the device is used without the balls, it becomes a stabilizer. However, the disclosed device is relatively complex in construction involving a tubular body having different thicknesses at opposite ends, and spherical balls attached by relatively flimsy stems that may rupture and result in one or more of the balls being lodged within the vaginal canal. The patentee contemplates the item to be made in different sized to accommodate all wearers. Also, the disclosed device including the soft protuberances in the form of attached balls does nothing to enhance or enlarge the girth of the penile member. Accordingly, while it may enhance the stimulation of the clitoris it does little or nothing to enhance the stimulation of the sensitive tissues within the vaginal canal, in the region of the crest commonly known as the g-spot or g-crest, which is believed to promote vaginal orgasms.

### SUMMARY OF THE INVENTION

Accordingly, it is an object of the invention to provide an enhancement device which overcomes the disadvantages of such prior art devices.

It is another object of the invention to provide an enhancement device which is simple in construction and economical to manufacture.

It is still another object of the invention to provide an enhancement device which promotes enhanced stimulation of the woman during vaginal intercourse without diminishing the sensitivity or pleasurable experience of the man.

It is yet another object of the invention to provide an enhancement device as in the previous objects which promotes both vaginal and clitoral orgasms in women.

It is a further object of the invention to provide an enhancement device which is made of a single or integral part which is simple to apply to the male penile member.

It is still a further object of the invention to provide an enhancement device of the type under discussion which can be used with conventional condoms.

It is yet a further object of the invention to provide an enhancement device as in the previous objects which avoids a construction that may cause or result in portions thereof inadvertently separating and being embedded in the vaginal canal.

It is an additional object of the invention to provide an enhancement device that can be made in one or more sizes, although one size can be used by all males.

It is still and additional object of the invention to provide an enhancement device that can substantially increase the girth of the penile member.

It is yet an additional object of the invention to provide an enhancement device as suggested which includes means for preventing the device from moving along the direction of the erectile penis during vaginal intercourse.

In order to achieve the above objects as well as others that will become evident from the disclosure that follows, a stimulation enhancement device in accordance with the invention comprises a generally elongate main body portion formed of a generally soft material and having predetermined length, width and thickness. Said main body portion defines an upwardly facing surface and an opposing downwardly facing surface. Said downwardly facing surface is provided with a generally flat surface and said upwardly facing surface is provided with a plurality of upwardly projecting protuberances rising above said upwardly facing surface. The downwardly facing surface of the device may be placed on the upper surface of an erect penis and when maintained thereon during vaginal intercourse the device effectively increases the girth of the penis while said protuberances are effective to provide vaginal stimulation.

### BRIEF DESCRIPTION OF THE DRAWINGS

These, and other objects will be readily evident from the description contained in the following specification, when taken together with the accompanying drawings, wherein:

FIG. 1 is a top plan view of a device in accordance with the invention;

FIG. 2 is a side elevational view of the device shown in FIG. 1; and

FIG. 3 is an enlarged cross-sectional view of the device taken along line 3-3 in FIG. 2, and further showing the device as positioned on the upper surface of an erect penile member to provide and increased girth and upwardly projecting protuberances.

### DESCRIPTION OF PREFERRED EMBODIMENTS

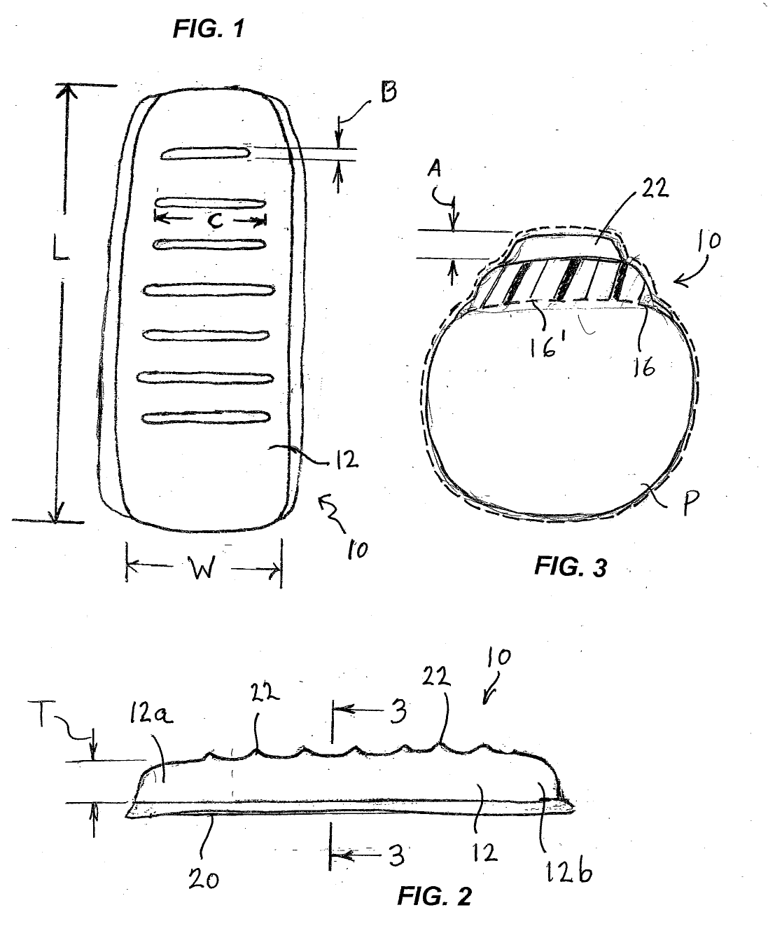

Referring now to the Figures, in which identical parts are designated by the same reference numeral throughout, and first referring to FIGS. 1 and 2, a stimulation enhancement device in accordance with the invention is generally designated by the reference numeral 10.

The device 10 includes a generally elongate main body portion 12 preferably formed of a generally soft, deformable material such as latex, rubber or other soft deformable material. The body portion 12 is made as a unitary member formed, for example by molding.

The body portion 12 has a front or remote end 12a and a rear or proximate end 12b. The body portion 12 defines an upwardly-directed or facing surface 14 and an opposing downwardly-directed or facing surface 16. All these directions or relative orientations are used for facility of description and are applicable when the device 10 is placed on and supported on an erect penis P (FIG. 3). While the lower surface 16 may be generally flat, it may also be somewhat curved inwardly along its length, as suggested at 16′ in FIG. 3 to more closely conform to the corresponding curvature of the erect member when positioned thereon.

Along each longitudinal edge of the body portion 12 there are provided flanges 18, 20 which are preferably relatively thin and flare outwardly as shown in FIG. 3, to avoid sharp or other edges that may rub against the skin and create any irritation or discomfort.

The main body portion has predetermined length L, width W and thickness T. While the specific dimensions are not critical they are preferably selected to generally cover the entire or substantially the entire surface of an erect penis. Thus, the length L may be 4″, although can generally be selected from the range of 3 to 5.5″; the width W may be 1.5″, although can generally be selected from the range of 1 to 2″; and the thickness T maybe 0.75″, although can generally be selected from the range of 0.5 to 1″. As evident from FIG. 3, the greater the thickness T and the width W the greater the cross-sectional area of the main body portion and the greater the girth created when the device is positioned as shown.

A feature of the invention is the provision of at least one but preferably a plurality of protuberances rising above the upper surface 14. In the illustrated preferred embodiment, the upwardly projecting protuberances are in the form of a plurality of generally transverse upwardly projecting ridges or bumps 22 that are substantially equally spaced from each other along the length of the main body portion 12. The specific shapes or dimensions “A”, “B” and “C” of the ridges are not critical, although these should be dimensioned and configured, when used to increase the overall girth, to stimulate both the clitoris as well as the g-spot within the vaginal canal.

The device 10 may be maintained in the desired position shown by any suitable means. A condom 24 closed on the end can used in the conventional way with the device enclosed within the condom. An open condom or tubular sleeve may also be used.

As is evident, therefore, the device 10 adds to the girth of the penis without diminishing sensitivity. However, because of the increased girth and the upwardly projecting protuberances or bumps 22 clitoral and vaginal stimulation is enhanced in the woman during vaginal intercourse.

After placing the device on top of an erect penis, positioned between the base and the head of the penis, a condom can be rolled over both the penis and the device while the device is in place, as suggested, in the normal or conventional way. After use, the device may optionally be washed and re-used. As indicated, the device may also be held in place by means of a headless condom. Clearly, a headless condom would not give the protection against sexually transmitted diseases or pregnancy.

The present disclosure describes presently preferred embodiments. However, the invention is not limited to those embodiments. Other variations are contemplated to be within the spirit and scope of the invention as defined in the appended claims.

1. A stimulation enhancement device comprising a generally elongate main body portion having predetermined length, width and thickness, said main body portion defining an upwardly-facing surface and an opposing downwardly-facing surface, said downwardly facing surface being generally flat and said upwardly-facing surface being provided with at least one upwardly-projecting protuberance rising above said upwardly-facing surface, whereby the downwardly-facing surface of the device may be positioned on the upper surface of an erect penis and, when maintained thereon, effectively increases the girth of the penis while said at least one protuberance is effective to provide enhanced vaginal and clitoral stimulation.

2. A stimulation enhancement device as defined in claim 1, wherein said main body portion is molded from a generally soft and deformable material.

3. A stimulation enhancement device as defined in claim 2, wherein the material is latex.

4. A stimulation enhancement device as defined in claim 2, wherein the material is rubber.

5. A stimulation enhancement device as defined in claim 1, wherein a plurality of protuberances are provided.

6. A stimulation enhancement device as defined in claim 5, wherein said protuberances are generally equally spaced from each other along the length direction of said main body portion.

7. A stimulation enhancement device as defined in claim 1, wherein said predetermined length is selected from the range of 3-5.5″.

8. A stimulation enhancement device as defined in claim 7, wherein said predetermined length is approximately 4.5″.

9. A stimulation enhancement device as defined in claim 1, wherein said predetermined width is selected from the range of 1-2″.

10. A stimulation enhancement device as defined in claim 9, wherein said predetermined width is approximately 1.5″.

11. A stimulation enhancement device as defined in claim 1, wherein said predetermined thickness is selected from the range of 0.5-1″.

12. A stimulation enhancement device as defined in claim 11, wherein said predetermined thickness is approximately 0.75″.

13. A stimulation enhancement device as defined in claim 1, further comprising securing means for securing the device on the upper surface of an erect penis.

14. A stimulation enhancement device as defined in claim 13, wherein said securing means comprises a condom closed at one end to cover the head of the penis.

15. A stimulation enhancement device as defined in claim A stimulation enhancement device as defined in claim 13, wherein said securing means comprises a condom open at a remote end to expose the head of the penis while being wrapped around the penis and device positioned thereon.

16. A stimulation enhancement device as defined in claim 1, wherein said downwardly-facing surface is curved to provide a concave surface configured and dimensioned to generally conform to the curvature of the erect penis when positioned thereon.

17. A stimulation enhancement device as defined in claim 1, wherein said at least one protuberance extends generally transversely to the longitudinal direction of said main body portion.

18. A stimulation enhancement device as defined in claim 17, wherein the width of said at least one protuberance is equal to at least one half of said predetermined width.
